(19) United States
(12) Patent Application Publication
(10) Pub. No.: US 2010/0068729 A1
(43) Pub. Date: Mar. 18, 2010

(54) METHOD AND KITS FOR THE DIAGNOSIS OF DIABETES

(75) Inventors: Samuel T. LABRIE, Austin, TX (US); Michael D. Spain, Austin, TX (US); James P. Mapes, Austin, TX (US); Ralph L. McDade, Austin, TX (US); Kenneth A. Pass, Glenmont, NY (US)

Correspondence Address: FOLEY AND LARDNER LLP;SUITE 500, 3000 K STREET NW, WASHINGTON, DC, 20007, US

(73) Assignees: Rules-Based Medicine, Inc.; Health Research, Inc.

(21) Appl. No.: 12/556,269

(22) Filed: Sep. 9, 2009

Related U.S. Application Data
(60) Provisional application No. 61/096,606, filed on Sep. 12, 2008.

(51) Int. Cl. G01N 33/53

(52) U.S. Cl. 435/7.1

(57) ABSTRACT

Provided are methods for the detection and diagnosis of a predisposition for developing diabetes. The methods are based on the discovery that abnormal levels of the selected analyte in biological samples, typically blood samples, of patients who are at risk are supportive of a diagnosis of a predisposition for developing diabetes. At least one new biomarker for a predisposition for diabetes is thus disclosed, IGF-1. Other important biomarkers for diabetes are described, including but not limited to ZAG, clusterin, corticosteroid-binding globulin, lumican, and serotransferrin. Kits containing reagents to assist in the analysis of biological samples are also described.

### CROSS REFERENCE TO RELATED APPLICATIONS

This application claims priority to U.S. Provisional Application No. 61/096,606, filed Sep. 12, 2008, the entire disclosure of which is incorporated herein by reference.

### FIELD OF THE INVENTION

Methods, kits and reagents for detection and/or diagnosis of patients with a predisposition for developing diabetes.

### DESCRIPTION OF THE RELATED ART

Diabetes mellitus (“diabetes”) is one of the leading causes of morbidity and mortality in the United States. Patients suffering from diabetes have metabolism disorder(s) and abnormally elevated blood sugar levels (i.e., hyperglycemia), which results from low levels of insulin production.

Beta cells found in the islets of Langerhans in the pancreas (“pancreatic islet cells) produce insulin. In Type I and (to a lesser extent) Type II diabetes, a deficiency in insulin production can be attributed to inadequate mass of functional insulin-producing islet cells. The cause of this cell loss may be viral, chemical, and/or autoimmune attack and destruction of the cells.

Individuals afflicted with diabetes can suffer from severe complications, including kidney damage, blindness, limb amputations, and cardiovascular disease. With early detection, many of these risks can be mitigated.

Early diagnosis allows for early treatment. Early treatment not only mitigates life-changing organ damage, but can also mitigate the cost of controlling future symptoms. For children, in particular detecting a high risk for diabetes will allow parents to monitor their children to prevent organ damage and other problems related to diabetes. For example, some studies have suggested certain measures such as breast-feeding, or giving children a 2000 IU of Vitamin D during their first year reduced the risk of type-1 diabetes. Thus, there is a critical need to develop additional biomarkers for early detection of diabetes. The present invention provides a method to diagnose persons with a propensity for diabetes.

### SUMMARY OF THE INVENTION

A method for rapid detection and/or accurate diagnosis of a predisposition for developing diabetes is provided. The method can be practiced with a determination of the concentrations of one biomarker in a patient biological sample. Elevated levels of the one biomarker, which is statistically different from levels found in “normals” (that is, control subjects not predisposed towards developing diabetes), support a positive diagnosis of a predisposition for developing diabetes. Preferably, the method utilizes an analyte or “biomarker” of a substance found in a biological sample (e.g., whole blood, dried blood, serum, plasma, or urine), to help support a positive or negative diagnosis of a predisposition for developing diabetes. Up to 70% accuracy in making a correct diagnosis is provided by the method.

According to the invention a method of diagnosing diabetes in a human subject, preferably a newborn, which comprises: (a) obtaining a biological sample from a human subject; (b) determining the concentration of IGF-1 in said sample; (c) deciding if the determined concentration of IGF-1 in said sample is statistically different from that found in a control group of human subjects, whereby a statistically different elevated concentration of IGF-1 supports a positive diagnosis of a pre-disposition for developing diabetes. Any one of a number of biological samples can be tested. Preferably, the fluid sample is selected from whole blood, dried blood, plasma, serum, or urine. It has been discovered that a measured concentration of about 6 ng/mL or above of IGF-1 in the biological sample supports a positive diagnosis of a predisposition for developing diabetes.

In another aspect of the invention a method is provided for screening for a predisposition of diabetes in a human subject, preferably a newborn, which method comprises: (a) obtaining a biological sample from a human subject; (b) determining the concentration of IGF-1 in said sample; (c) deciding if the determined concentration of IGF-1 in said sample is statistically different from that found in a control group of human subjects, whereby a statistically different elevated concentration of IGF-1 supports a positive diagnosis of a predisposition for developing diabetes. It has been found that a measured concentration of about 3 ng/mL or above of IGF-1 in said sample supports a positive diagnosis of diabetes.

Various techniques for assessing the importance of a certain biomarker in arriving at a diagnosis is also described herein. One such technique includes the application of one or more statistical methods (e.g., linear regression analysis, classification tree analysis, heuristic nave Bayes analysis and the like).

Also provided is a kit comprising reagents for determining the concentration in a biological sample of a panel of analytes including IGF-1. The reagents may include antibodies against the members of a given panel of analytes. Furthermore, the reagent may be immobilized on a substrate, which substrate may comprise a two-dimensional array, a microtiter plate, or multiple bead sets.

The methods may further comprise comparing the levels of the one, two, or more biomarkers in a patient's biological sample with levels of the same biomarkers in one or more control samples by applying a statistical method such as: linear regression analysis, classification tree analysis and heuristic nave Bayes analysis. The statistical method may be, and typically is performed by a computer process, such as by commercially available statistical analysis software. In one embodiment, the statistical method is a classification tree analysis, for example CART (Classification and Regression Tree).

An article of manufacture is provided which comprises binding reagents specific for at least IGF-1. More preferably, a kit is provided which comprises binding reagents specific for IGF-1. In a preferred embodiment, each binding reagent is immobilized on a substrate. For example, monoclonal antibodies against IGF-1 and the other biomarkers described herein are immobilized independently to one or more discrete locations on one or more surfaces of one or more substrates. The substrates may be beads comprising an identifiable biomarker, wherein each binding reagent is attached to a bead comprising a different identifiable biomarker than beads to which a different binding reagent is attached. The identifiable biomarker may comprise a fluorescent compound, a quantum dot, or the like.

In a further embodiment, a method of predicting onset of diabetes is provided, comprising determining the change in concentration at two or more points in time of one or more markers in a patient's biological sample, wherein an observed increase in the concentration of IGF-1 in the patient's biological sample between the two time points, is predictive of the onset of diabetes.

Other aspects of the invention will become apparent to those of ordinary skill after considering the detailed descriptions provided herewith.

### DETAILED DESCRIPTION OF THE PREFERRED EMBODIMENTS

The use of numerical values in the various ranges specified in this application, unless expressly indicated otherwise, are stated as approximations as though the minimum value within the stated preceded by the word “about.” In this manner, slight variations above and below the stated ranges can be used to achieve substantially the same results as values within the ranges. Also, the disclosure of these ranges is intended as a continuous range including every value above the minimum value.

Provided herein is an assay for rapid identification of a patient who is predisposed towards developing diabetes. Preferably, the patient is a child. A patient that is “predisposed” to develop diabetes or is referred to as “pre-diabetic” is a patient that is more likely than a standard control group comprised of the general population to develop diabetes in the future. Identified below are certain biological sample (e.g., blood) analytes or biomarkers useful in the detection and/or diagnosis of diabetes. The biomarker IGF-1 has been found to be over-expressed in patients who will eventually be diagnosed with diabetes.

The parameters for establishing the significance of one biomarker for the diagnosis of diabetes is determined statistically by comparing normal or control blood (preferably, e.g., whole blood, dried blood, serum or plasma) levels of this biomarker with blood levels in patients clinically and properly diagnosed as having a predisposition for developing diabetes. As a non-limiting example of estimates of significant threshold values in support of a positive diagnosis of diabetes, the following concentrations are provided: IGF-1 (about 6 ng/mL or above) for diagnosing a predisposition to develop diabetes, or IGF-1 (about 3 ng/mL or above) for screening for a predisposition to develop diabetes.

It is understood that these values are approximate. Statistical methods can be used to define the critical range of values. Typically within one standard deviation of those approximate values might be considered as statistically significant values for determining a statistically significant difference, preferably two standard deviations. For this reason, the word “about” is used in connection with the stated values. “Statistical classification methods” are used to identify biomarkers capable of discriminating normal patients from patients with diabetes and are further used to determine critical blood values for each biomarker for discriminating between such patients. Certain statistical methods can be used to identify discriminating biomarkers and panels thereof. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS™, commercially available from Insightful Corporation of Seattle, Wash.

By identifying biomarkers useful in the determination and/or diagnosis of diabetes and by use of statistical methods to identify which biomarkers and groups of biomarkers are particularly useful in identifying diabetes-at-risk patients, a person of ordinary skill in the art, based on the disclosure herein, can compose panels of biomarkers having superior selectivity and sensitivity. Examples of biomarkers that can be included in panels, which provide excellent discriminatory capability, include: IGF-1, zinc-α-2-glycoprotein 1 (ZAG), clusterin, corticosteroid-binding globulin, lumican, and serotransferrin. It will be recognized by those of ordinary skill in the field of biostatistics, that the number of biomarkers in any given panel may be different depending on the combination of biomarkers. With optimum sensitivity and specificity being the goal, one panel may include two biomarkers, another may include five, and still others may include twelve or more, yielding similar results.

The invention is based on an evaluation of IGF-1 levels, alone and/or in combination with levels of other biomarkers, in blood for diagnosis of diabetes in all stages of it progression. Patients with diabetes are at considerable risk for heart disease and stroke, high blood pressure, blindness, kidney disease, nervous system disease, amputations, dental disease, pregnancy complications and other serious complications, and outcomes can be improved with appropriate diagnosis and therapy. Thus, rapid and accurate diagnosis of patients predisposed to developing diabetes is critical for patient care.

The results described herein demonstrate that serum IGF-1 levels are elevated in patients predisposed for developing diabetes. Thus, IGF-1 can be used as an early biomarker of diabetic conditions, and in particular, diabetes.

The present method includes measuring the level of IGF-1 in a biological sample (e.g., blood spots, whole blood, plasma, serum or urine and the like) from a patient; comparing the respective levels with that of control subjects; and diagnosing the state of disease based on the level of IGF-1 relative to that of control subjects. A patient can be diagnosed with predisposition for developing diabetes if the level of IGF-1 is increased relative to that of control subjects.

Analyte levels can be measured using an immunoassay such as an ELISA or a multiplexed method as described below, and in more detail by Chandler et al., U.S. Pat. No. 5,981,180 (Luminex Corporation).

IGF-1 levels above about 3 ng/mL were identified in pre-diabetic patients. Without being bound by a particular mechanism, IGF-1 may be directly involved in the pathophysiology of diabetes. For example, IGF-1 has similarity to Growth Hormone in countering insulin so as to maintain glucose homeostasis. It is possible that IGF-1 may be elevated in infants with a predisposition to diabetes because these infants may have a phase where they oversecrete insulin before being insulin depleted.

The analytes used in the method of the invention can be detected, for example, by a binding assay. For example, a sandwich immunoassay can be performed by capturing IGF-1 from a biological sample with antibodies having specific binding affinity for each protein, which then can be detected with a labeled antibody having specific binding affinity for the analyte. Alternatively, standard immunohistochemical techniques can be used to detect IGF-1 using such antibodies. Antibodies having affinity for IGF-1 are available.

The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fᵥ fragments; single chain Fᵥ (scFᵥ) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fᵥ fragments, scFᵥ tandems ((scFᵥ)₂ fragments), diabodies, tribodies or tetrabodies, which typically are covalently linked or otherwise stabilized (i.e., leucine zipper or helix stabilized) scFᵥ fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475096, 5,840867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available from BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood biomarker, namely at least IGF-1, or any combination of the foregoing.

Data generated from an assay to determine blood levels of IGF-1 can be used to determine the likelihood of a patient developing diabetes. As shown herein, if the following condition is met in a patient's blood, IGF-1 (about 3 ng/mL or above for a screen or 6 ng/mL or above), there is a very high likelihood that the patient has an elevated risk of developing diabetes. In one embodiment, an elevated IGF-1 level, relative to the level of the biomarker of interest in a population of normal or control patients, indicates the existence of a elevated chance for developing diabetes in the patient. (See, Table 2, discussed further elsewhere herein.)

In the context of the present disclosure, “blood” includes any blood fraction, for example serum, that can be analyzed according to the methods described herein. Serum is a standard blood fraction that can be tested, however, IGF-1 may be detected from any blood fraction. By measuring blood levels of a particular biomarker, it is meant that any appropriate blood fraction can be tested to determine blood levels and that data can be reported as a value present in that fraction. As a non-limiting example, the blood levels of a biomarker can be presented as 50 ng/mL serum.

### Example I

Patient Population. The patient population consists of diabetic children, 2-3 years of age, born in New York State, and therefore had a sample of their blood collect and stored shortly after birth. New York collects and stores blood spot samples from infants born in the state. The children developed type 1 diabetes around the age of 2 or 3. (Type 1 diabetes is usually diagnosed in children and young adults. In individuals with type 1 diabetes, the body does not produce insulin, the hormone needed to convert glucose, starches, and other foods into energy).

In analyzing the blood spots of the diabetic children that were taken at birth, the present inventors found an IGF-1 level of these “pre-diabetic” children was significantly higher at birth than the IGF-1 level of the general population at birth.

Development of Luminex assay. The reagents for multiplex system were developed using antibody pairs purchased from R&D Systems (Minneapolis, Minn.), Fitzgerald Industries International (Concord, Mass.) or produced by well known immunological methods. Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiague, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μm of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG (BD Biosciences, San Diego, Calif.). Detection Abs were biotinylated using EZ-Link Sulfo-NHS-Biotinylation Kit (Pierce, Rockford, Ill.) according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for patient samples and measured twice at two different time points. The intra-assay variability within the replicates is expressed as an average coefficient of variation. Inter-assay variability was evaluated by testing quadruplicates of each standard and sample with an average of 16.5% (data not shown). Newly developed kits were multiplexed together and the absence of cross-reactivity was confirmed according to Luminex protocol.

Examples of some commercial sources of matched antibody cytokine pairs include MAB636 EGF (R&D Systems, Minneapolis, Minn.), BAF236 G-CSF (R&D Systems), DY214 IL-6 (R&D Systems), DY206 IL-8 (R&D Systems), DY208 IL-12p40 (R&D Systems), DY1240 MCP-1 (R&D Systems), DY279 VEGF (R&D Systems), DY293 CA-125 (M002201, M002203, Fitzgerald Industries International, Inc., Concord, Mass.).

### Results

Concentrations of biomarkers were obtained by LabMap technology. Circulating concentrations of different biomarkers were evaluated in a multiplexed assay using LabMap technology in blood of patients from the pre-diabetic and control groups. Table 1 lists the data used to analyze the difference between the two groups. Patients with IGF-1 levels below the detectable threshold are designated as “low.” For purposes of analyzing the data, these “low” values were assigned a number that is 50% of the lowest accurate measurement (i.e., 0.24 ng/mL).

TABLE 1
IGF-1 levels of patients later diagnosed with diabetes
(pre-diabetic) and IGF-1 levels of a control group.

| | | |
|---|---|---|
| Sample # | IGF-1 level (ng/mL) | “Lows” Converted (ng/mL) |

Pre-Diabetic Data

| | | |
|---|---|---|
| 1 | 16 | 16 |
| 2 | 8.9 | 8.9 |
| 3 | 3.4 | 3.4 |
| 4 | 57 | 57 |
| 5 | <LOW> | 0.24 |
| 6 | 59 | 59 |
| 7 | 12 | 12 |
| 8 | 64 | 64 |
| 9 | 28 | 28 |
| 10 | <LOW> | 0.24 |
| 11 | 6.4 | 6.4 |
| 12 | 59 | 59 |
| 13 | 19 | 19 |
| 14 | 16 | 16 |
| 15 | 8.3 | 8.3 |
| 16 | 15 | 15 |
| 17 | 9.2 | 9.2 |
| 18 | 10 | 10 |
| 19 | 11 | 11 |
| 20 | 10 | 10 |
| 21 | 16 | 16 |
| 22 | 12 | 12 |
| 23 | 23 | 23 |
| 24 | 52 | 52 |
| 25 | 13 | 13 |
| 26 | 6.4 | 6.4 |
| 27 | 53 | 53 |
| 28 | 59 | 59 |
| 29 | <LOW> | 0.24 |
| 30 | 9.7 | 9.7 |
| 31 | 11 | 11 |
| 32 | 5.3 | 5.3 |
| 33 | 7.6 | 7.6 |
| 34 | 5.3 | 5.3 |
| 35 | 12 | 12 |
| 36 | 1.9 | 1.9 |
| 37 | 57 | 57 |
| 38 | 63 | 63 |
| 39 | 4.6 | 4.6 |
| 40 | 13 | 13 |
| 41 | 58 | 58 |
| 42 | 6.6 | 6.6 |
| 43 | 55 | 55 |
| 44 | <LOW> | 0.24 |
| 45 | 11 | 11 |
| 46 | <LOW> | 0.24 |
| 47 | 25 | 25 |
| 48 | 4.6 | 4.6 |

Control Group Data

| | | |
|---|---|---|
| 1 | 6.6 | 6.6 |
| 2 | 20 | 20 |
| 3 | 4.7 | 4.7 |
| 4 | 0.48 | 0.48 |
| 5 | 3.0 | 3.0 |
| 6 | 10.0 | 10.0 |
| 7 | 2.5 | 2.5 |
| 8 | 4.7 | 4.7 |
| 9 | 56 | 56 |
| 10 | 3.7 | 3.7 |
| 11 | 7.4 | 7.4 |
| 12 | 1.9 | 1.9 |
| 13 | 6.3 | 6.3 |
| 14 | 1.2 | 1.2 |
| 15 | 3.9 | 3.9 |
| 16 | 3.5 | 3.5 |
| 17 | 1.3 | 1.3 |
| 18 | 5.2 | 5.2 |
| 19 | 53 | 53 |
| 20 | 7.9 | 7.9 |
| 21 | <LOW> | 0.24 |

Table 2 illustrates the diagnostic accuracy obtained by testing for the analyte and determining how useful it would be as a diagnostic tool. Based on the cutoff concentration used, there is a sliding scale between the number of pre-diabetics detected, and the number of false positives. Using at cutoff at 6 ng/mL, 37 out of 48 pre-diabetics would have been identified (77% sensitivity), and 13 out of 21 members of the control group (62% specificity) would have been correctly identified as not-at-risk for developing diabetes. As the IGF-1 concentration cutoff is increased, fewer pre-diabetics are identified (lower sensitivity), but there are fewer false positives (higher specificity). Thus, a lower IGF-1 cutoff could be used as a screening test for pre-diabetes, whereas a higher cutoff would be useful for eliminating false positives—but may not catch all of the patients with a predisposition for developing diabetes.

TABLE 2
IGF-1 Predictive capacity at different concentrations
of pre-diabetic (PD) versus control group (N)

| Cutoff | # PD | | # N | |
|---|---|---|---|---|
| (ng/mL) | correct | % Sensitivity | correct | % Specificity |
| 3 | 42 | 88% | 7 | 33% |
| 4 | 41 | 85% | 10 | 48% |
| 5 | 39 | 81% | 12 | 57% |
| 6 | 37 | 77% | 13 | 62% |
| 7 | 34 | 71% | 15 | 71% |
| 8 | 33 | 69% | 17 | 81% |
| 9 | 31 | 65% | 17 | 81% |
| 10 | 29 | 60% | 18 | 86% |
| 11 | 24 | 50% | 18 | 86% |
| 12 | 23 | 48% | 18 | 86% |
| 13 | 19 | 40% | 18 | 86% |
| 14 | 19 | 40% | 18 | 86% |
| 15 | 18 | 38% | 18 | 86% |

Table 3 illustrates the statistical significance of IGF-1 as a marker for diagnosing the pre-diabetic state. The median IGF-1 value for pre-diabetic patients is 11.4, which is substantially different than the IGF-1 value of 4.7 found in the control group. Furthermore, the mean IGF-1 value for pre-diabetic patients is 20.8, which is substantially different than the value of 9.7 found in the control group. The differences in both the mean and median values are statistically significant.

TABLE 3
Comparison of Values in Pre-Diabetic
(PD) versus Control Group (N)

| | Median | Mean | Standard Deviation |
|---|---|---|---|
| Pre-Diabetic (PD) | 11.4 | 20.8 | 21.28 |
| Control (N) | 4.7 | 9.7 | 15.47 |

### Example II

### Development of LabMap Assays for Circulating Antibodies

Assays were performed in filter-bottom 96-well microplates (Millipore). Purified antigens of interest were coupled to Luminex beads as described for antibodies. Antigen-coupled beads were pre-incubated with blocking buffer containing 4% BSA for 1 h at room temperature on microtiter shaker. Beads were then washed three times with washing buffer (PBS, 1% BSA, 0.05% Tween 20) using a vacuum manifold followed by incubation with 50 μL blood serum diluted 1:250 for 30 min at 4 C. This dilution was selected as an optimal for recovery of anti-IL-18 IgG based on previous serum titration (data not shown). Next, washing procedure was repeated as above and beads were incubated with 50 μl/well of 4 μg/mL PE-conjugated antibody raised against human IgG for 45 min in the dark with the constant shaking. Wells were washed twice, assay buffer was added to each well and samples were analyzed using the Bio-Plex suspension array system (Bio-Rad Laboratories, Hercules, Calif.). For standard curve, antigen-coupled beads were incubated with serially diluted human antibodies against specific antigens. Purification of monospecific human antibodies is described above. Data analysis was performed using five-parametric-curve fitting.

In another particular aspect of the invention, the expression profiles of one or a plurality of the disclosed markers could provide valuable molecular tools for examining the molecular basis of drug responsiveness in diabetes and for evaluating the efficacy of drugs for diabetes or their side effects. Changes in the expression profile from a baseline profile while the cells are exposed to various modifying conditions, such as contact with a drug or other active molecules can be used as an indication of such effects.

Therefore, the invention provides a test for use in determining whether a patient suffering from diabetes will respond to therapy comprising the steps of, performing the diagnostic steps of the inventive method described hereinabove for body samples obtained respectively from an individual treated for diabetes with a pharmaceutically acceptable agent and an individual not diagnosed with diabetes, and determining the responsiveness to drug therapy.

Monitoring the influence of agents (e.g., drug compounds) on the level of expression of a marker of the invention can be advantageously applied in clinical trials. For example, the effectiveness of an agent to affect marker expression can be monitored in clinical trials of subjects receiving treatment for diabetes. In a preferred embodiment, the present invention provides a method for monitoring the efficacy of treatment of a subject with an agent (e.g., an agonist, antagonist, peptidomimetic, protein, peptide, nucleic acid, small molecule, or other drug candidate) comprising the steps of: (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly.

For example, modified administration of the agent can be desirable to increase expression of the novel diagnostic marker(s) to higher levels than detected, i.e., to increase the effectiveness of the agent. Alternatively, increased/decreased administration of the agent can be desirable to increase/decrease the effectiveness of the agent, respectively.

As used herein, the term “candidate agent” or “drug candidate” can be natural or synthetic molecules such as proteins or fragments thereof, antibodies, small molecule inhibitors or agonists, nucleic acid molecules, e.g., antisense nucleotides, ribozymes, double-stranded RNAs, organic and inorganic compounds and the like.

In another particular aspect of the present invention, a method is provided for both prophylactic and therapeutic methods of treating a subject having, or at risk of having, diabetes. Administration of a prophylactic agent can occur prior to the manifestation of symptoms characteristic of diabetes, such that development of the diabetes is prevented or delayed in its progression.

Examples of suitable therapeutic agents include, but are not limited to, antisense nucleotides, ribozymes, double-stranded RNAs, ligands, small molecules and antagonists as described more in detail below.

In a particular embodiment, the invention provides a method for treating or preventing diabetes in an individual comprising the step of administering to said individual a therapeutically effective amount of a modulating compound that modulates expression or activity of one or more of the genes, gene expression, or protein products of the group of the IGF-1 genes, so that at least one symptom of diabetes is ameliorated.

In another aspect, the invention provides a method for treating or preventing diabetes in an individual comprising the step of administering to said individual a therapeutically effective amount of a modulating compound that modulates expression or activity of one or more of the genes or gene expression products of the group of genes (e.g., ZAG, clusterin, corticosteroid-binding globulin, lumican, and serotransferrin) so that at least one symptom of diabetes is ameliorated.

In another particular aspect of the invention, by virtue of the differential expression of the inventive diagnostic markers, it is possible to utilize these markers to enhance the certainty of prediction of whether a particular drug treatment in a patient will be effective in treating diabetes. Therefore, the invention provides a method for identifying candidate agents for use in the treatment of diabetes comprising the steps of: a) contacting a sample of tissue subject to toxicity with a candidate agent; b) determining from the tissue the level of gene or protein expression corresponding to the IGF-1 gene, to obtain a first set of value; and c) comparing the first set of value with a second set of value corresponding to the level of gene expression, assessed for the same gene (s) and under identical condition as for step b) in a tissue subject to toxicity not induced by the candidate agent, wherein a first value substantially equal or lower than the second value for IGF-1 expression is an indication that the candidate agent is ameliorating symptoms of diabetes. Similarly, a first value substantially equal or higher than the second value for at least one of IGF-1 expression is an indication that the candidate agent is exacerbating symptoms of diabetes or has not effect thereon.

In another particular aspect of the invention, a method is provided for identifying candidate agents for use in the treatment of diabetes comprising the steps of (a) contacting a sample of a tissue subject to toxicity with a candidate agent; (b) determining from the tissue the level of gene or protein expression corresponding to IGF-1, to obtain a first set of value(s); and (c) comparing the first set of values with a second set of values corresponding to the level of expression assessed for the same gene(s) and under identical condition as for step b) in a tissue subject to toxicity not induced by the candidate agent wherein a first value substantially greater than the second value for said gene expression is an indication that the candidate agent is ameliorating symptoms of diabetes.

In a preferred embodiment, the means for determining the level of gene expression comprises oligonucleotides specific for a marker gene. Particularly preferred are methods selected from Northern blot analysis, reverse transcription PCR or real time quantitative PCR, branched DNA, nucleic acid sequence based amplification (NASBA), transcription-mediated amplification, ribonuclease protection assay, and microarrays. Sequences of the marker genes disclosed herein are readily available from publicly accessible gene banks, such as GENBANK, and for sake of conciseness, are not detailed here.

Particularly useful methods for detecting the level of mRNA transcripts obtained from the novel markers disclosed herein include hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can, in another embodiment, be compared with the gene expression profile derived form the sample obtained from the disease-free subject, and thereby determine whether the subject has or is at risk of developing diabetes.

The gene expressions of the markers can also be preferably assessed in the form of a kit using RT-PCR, a high throughput technology: The well-known technique RT-PCR reaction exploits the 5′ nuclease activity of AmpliTaqGold DNAPolymerase to cleave a TaqMan probe during PCR. The probe consists of an oligonucleotide (usually >20 mer) with a 5′-reporter dye and a 3′-quencher dye. The fluorescent reporter dye, such as FAM (6-carboxyfluorescein), is covalently linked to the 5′ end of the oligonucleotide. The reporter is quenched by TAMRA (6-carboxy-N, N, N′,N′-tetramethylrhodamine) attached via a linker arm that is located at the 3′ end.

Oligonucleotide probes used for each marker should derive from the nucleotide sequence of the gene of such marker, the selection of the appropriate oligonucleotide sequence being now a matter of standard routine technique for one skilled in the art. Again, because the DNA sequences of the respective genes described herein are freely available, they have not be reiterated here.

In addition to the drug screening methods known, cell-free assays can also be used to identify compounds which are capable of interacting with proteins encoded by the markers taught herein (e.g., IGF-1), to alter the activity of the protein or its binding partner. Cell-free assays can also be used to identify compounds, which modulate the interaction between the encoded protein and its binding partner such as a target peptide.

In one embodiment, cell-free assays for identifying such compounds comprise a reaction mixture containing a marker protein and a test compound or a library of test compounds in the presence or absence of the binding partner, e.g., a biologically inactive target peptide, or a small molecule. Interaction between molecules can also be assessed by using real-time BIA (Biomolecular Interaction Analysis, Pharmacia Biosensor (AB) which detects surface plasmon resonance, an optical phenomenon. Formation of a complex between the protein and its binding partner can be detected by using detectably labeled proteins such as radiolabeled, fluorescently labeled, or enzymatically labeled protein or its binding partner, by immunoassay or by chromatographic detection.

In another embodiment, activity of a target RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides).

As discussed above, antisense nucleotides can be delivered to cells, which express the described genes in vivo by various techniques, e.g., injection directly into tissue, entrapping the antisense nucleotide in a liposome, by administering modified antisense nucleotides which are targeted to the heart cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in an alternative embodiment, the nucleic acid comprising an antisense nucleotide sequence is placed under the transcriptional control of a promoter, i.e., a DNA sequence which is required to initiate transcription of the specific genes, to form an expression construct. The antisense nucleic acids of the invention are controllably expressed intracellularly by transcription from an exogenous sequence. If the expression is controlled to be at a high level, a saturating perturbation or modification results. In conclusion, antisense nucleic acids can be routinely designed to target virtually any mRNA sequence including the marker genes cited in the present document, and a cell can be routinely transformed with or exposed to nucleic acids coding for such antisense sequences such that an effective and controllable or saturating amount of the antisense nucleic acid is expressed. Hence, the translation of virtually any RNA species in a cell can be modified or perturbed.

In addition, the activities of marker proteins can be modified or perturbed in a controlled or a saturating manner by exposure to exogenous drugs or ligands. Since the methods of this invention are often applied to testing or confirming the usefulness of various drugs to treat diabetic disorders, drug exposure is an important method of modifying/perturbing cellular constituents, both mRNA's and expressed proteins.

In a preferable case, a drug is known that interacts with only one marker protein in the cell and alters the activity of only that one marker protein, either increasing or decreasing the activity. Graded exposure of a cell to varying amounts of that drug thereby causes graded perturbations of network models having that marker protein as an input. Saturating exposure causes saturating modification/perturbation.

The term “antagonist” refers to a molecule which, when bound to the protein encoded by the gene, inhibits its activity. Antagonists can include, but are not limited to, peptides, proteins, carbohydrates, and small molecules. In a particularly useful embodiment, the antagonist is an antibody specific for IGF-1. The antibody alone may act as an effector of therapy or it may recruit other cells to actually effect cell killing.

In the case of treatment with an antisense nucleotide, the method comprises administering a therapeutically effective amount of an isolated nucleic acid molecule comprising an antisense nucleotide sequence derived from at least one marker identified hereinabove wherein the antisense nucleotide has the ability to change the transcription/translation of the at least one gene. In the case of treatment with an antagonist, the method comprises administering to a subject a therapeutically effective amount of an antagonist that inhibits or activates a protein encoded by at least one marker identified above.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat diabetes. The determination of a therapeutically effective amount is well within the capability of those skilled in the art. For any therapeutic, the therapeutically effective dose can be estimated initially either in cell culture assays, e.g., of neoplastic cells, or in animal models, usually rats, mice, rabbits, dogs or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutically effects is the therapeutic index, and it can be expressed as the ratio LD50/ED50. Antisense nucleotides, ribozymes, double-stranded RNAs and antagonists that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage may be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight and gender of the subject, diet, time and frequency of administration, drug combination(s), reaction sensitivities, and tolerance/response to therapy.

Normal dosage amounts may vary form 0.1 to 100,000 micrograms, up to a total dosage of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone or in combination with other agents, drugs or hormones.

The pharmaceutical compositions may be administered by a number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredient, these pharmaceutical compositions may contain suitable pharmaceutically acceptable carriers comprising excipients and auxiliaries which facilitate processing of the active compounds into preparations which can be used pharmaceutical.

Pharmaceutical compositions for oral administration can be formulated using pharmaceutical acceptable carriers well known in the art in dosages suitable for oral administration. Such carriers enable the pharmaceutical compositions to be formulated as tablets, pills, capsules, liquids, gels, syrups, slurries, suspensions, and the like, for ingestion by the patient.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

What is claimed is:

1. A method of identifying a human subject having a predisposition for developing diabetes, comprising:
(a) obtaining a biological sample from a human subject;
(b) determining the concentration of IGF-1 in said biological sample;
(c) deciding if the determined concentration of IGF-1 in said biological sample is statistically different from that found in a control group of human subjects,
whereby a statistically different elevated concentration of IGF-1 supports a positive diagnosis of a predisposition for developing diabetes.

2. The method of claim 1 in which said human subject is complaining about or shows signs of a plurality of the following symptoms: increased thirst, impaired visibility, increased urination, or unexplained weight loss, weakness, fatigue, tingling in limbs, numbness in limbs, dry or itchy skin, frequent infections, bruises that are slow to heal.

3. The method of claim 1 in which said biological sample is selected from the group consisting of whole blood, dried blood, plasma, serum, or urine.

4. The method of claim 1 in which a determined concentration of about 6 ng/mL or above of IGF-1 in said biological sample supports a positive diagnosis.

5. The method of claim 1 in which concentrations are determined by conducting one or more immunoassays.

6. The method of claim 1 which includes applying a statistical method selected from the group consisting of linear regression analysis, classification tree analysis and heuristic nave Bayes analysis.

7. A method of screening a human subject for having a predisposition for developing diabetes, comprising:
(a) obtaining a biological sample from a human subject;
(b) determining the concentration of IGF-1 in said biological sample;
(c) deciding if the determined concentration of IGF-1 in said biological sample is statistically different from that found in a control group of human subjects,
whereby a statistically different elevated concentration of IGF-1 supports a positive diagnosis of screening for a predisposition for developing diabetes.

8. The method of claim 7 in which said biological sample is selected from the group consisting of whole blood, dried blood, plasma, serum, or urine.

9. The method of claim 7 in which a determined concentration of about 3 ng/mL or above of IGF-1 in said biological sample supports a positive diagnosis.

10. The method of claim 7 in which concentrations are determined by conducting one or more immunoassays.

11. A method of diagnosing diabetes in a human subject suspected of suffering from diabetes, comprising:
(a) obtaining a biological sample from a human subject;
(b) determining the concentration of IGF-1 in said biological sample;
(c) deciding if the determined concentration of IGF-1 in said biological sample is statistically different from that found in a control group of human subjects,
whereby a statistically different elevated concentration of IGF-1 supports a positive diagnosis of a predisposition for developing diabetes.

12. The method of claim 11 in which said biological sample is selected from the group consisting of whole blood, dried blood, plasma, serum, or urine.

13. The method of claim 11 in which a determined concentration of about 6 ng/mL or above of IGF-1 in said biological sample supports a positive diagnosis.

14. The method of claim 11 in which concentrations are determined by conducting one or more immunoassays.

15. A kit comprising reagents for determining the concentration in a biological sample of a panel of analytes including IGF-1.

16. The kit of claim 15 which includes antibodies against a panel of analytes including IGF-1.

17. The kit of claim 15 which includes reagents immobilized on a substrate.

18. The kit of claim 17 which the substrate comprises a two-dimensional array, a microtiter plate, or multiple bead sets.
